(19) United States
(12) Patent Application Publication
(10) Pub. No.: US 2024/0389916 A1
(43) Pub. Date: Nov. 28, 2024

(54) RECOGNIZING UNHEALTHY STRESS IN PUBLIC SAFETY WORKERS AND TAKING ACTION TO REMEDY

(71) Applicant: INTRADO LIFE & SAFETY, INC., Longmont, CO (US)

(72) Inventors: Adan K. Pope, Riverside, IL (US); Michel Brkovic, St-Eustache (CA); Candace D. Allred, Whitsett, NC (US)

(21) Appl. No.: 18/201,613

(22) Filed: May 24, 2023

(51) Int. Cl. A61B 5/16; A61B 5/021; G16H 50/20

(52) U.S. Cl. CPC A61B 5/165; A61B 5/021; G16H 50/20

(57) ABSTRACT

A method includes observing a call taker to produce first data, determining a first stress level of the call taker, at least in part based on the first data, determining if the first stress level exceeds a first predetermined threshold, determining an output action, if the first stress level exceeds the first predetermined threshold, and notifying of the action.

### BACKGROUND

### Technical Field

This disclosure relates to centralized call answering arrangements and, in particular, to arrangements to reduce the stress levels of call takers in such arrangements.

### Related Art

Today, public safety call takers in Emergency Communication Centers (ECCs) undergo unhealthy stress levels due to the nature of the occupation. Constant time pressure, severity of real-life incidents, and sedentary work all contribute to staff turnover and poor health, both mental and physical.

### BRIEF SUMMARY

In one implementation of the present disclosure, a method includes observing a call taker to produce first data; determining a first stress level of the call taker, at least in part based on the first data; determining if the first stress level exceeds a first predetermined threshold; determining an output action, if the first stress level exceeds the first predetermined threshold; and notifying of the action.

In another implementation of the present disclosure, a method to be implemented in a communication center includes: observing a call taker to provide first data; determining a first stress level of the call taker, at least in part based on the first data; determining if the first stress level exceeds a first predetermined threshold; determining an output action, if the first stress level exceeds the first predetermined threshold; and notifying of the action.

### BRIEF DESCRIPTION OF THE SEVERAL VIEWS OF THE DRAWINGS

FIG. 1 illustrates an exemplary call center, according to an implementation of the present disclosure.

FIG. 2 illustrates an exemplary stress recognition algorithm, according to an implementation of the present disclosure.

FIG. 3 illustrates suboperations of an initialization operation, according to an implementation of the present disclosure.

FIG. 4 illustrates suboperations of an action output operation, according to an implementation of the present disclosure.

FIG. 5 illustrates an algorithm for call taker behavior modification, based on big data collection, according to an implementation of the present disclosure.

FIG. 6 illustrates a computing device, according to an implementation of the present disclosure.

### DETAILED DESCRIPTION

Disclosed are a method and system for recognizing unhealthy stress in public safety workers and recommending corrective action with closed loop feedback. In various implementations, a system measures and understands the indication of an unhealthy work environment, recommends actions, and/or makes environmental changes. The system can improve the working condition for ECC call takers, create instant gratification rewards to encourage healthy behaviors, and then measure the response post-actions-taken for continual improvement. Thus, potential benefits of particular implementations include ECC call taker retention, improved health of call takers, and reducing the need for extended hours of call takers to cover missed shifts.

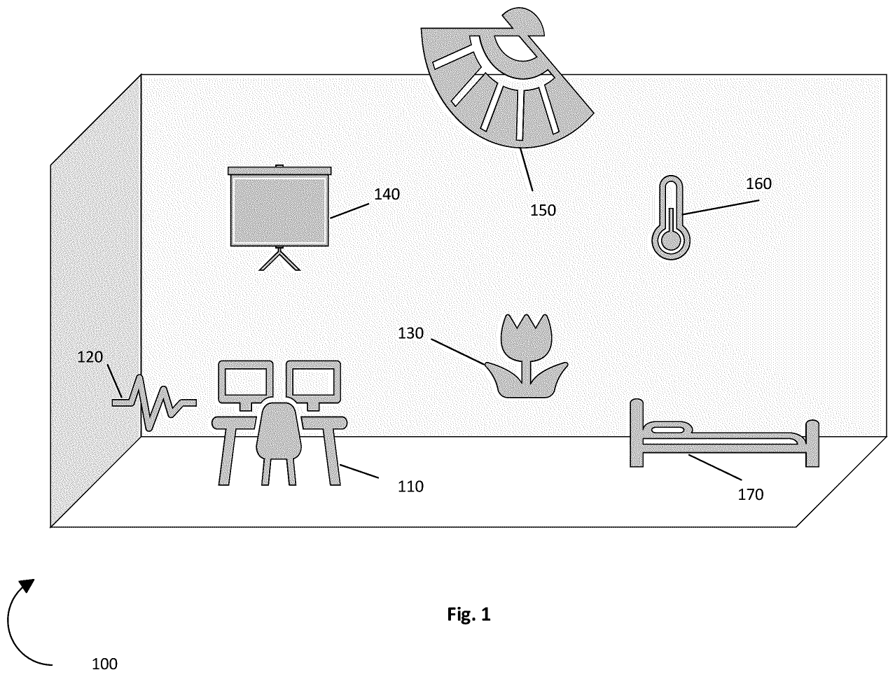

FIG. 1 illustrates an exemplary call center 100, according to an implementation of the present disclosure. The call center 100 includes a workstation 110, a sensor 120, an electronic air freshener (e.g., Glade PlugIn) 130, a display 140, a fan 150, a temperature control 160, and a cot 170.

A call taker sits at the workstation 110 and uses a computer (including a display) and a telephone line to address emergency calls in a telephone queue. In many implementations, the computer is connected to the Internet. In a contemporary call center, there often are two or more incoming telephone lines. Thus, the incoming calls are shared in a telephone queue. While a call taker is on shift, the telephone queue generally assigns the incoming calls to each of the call takers in a round robin method. Generally, the content of each incoming call is unknown, as the call taker is the first point-of-contact for the caller. Thus, a call taker can receive several calls about violent emergencies in a row, contributing to significant stress on the call taker.

The sensor 120 can include one or more biometric sensors to measure aspects of the call taker. The biometric sensors can be or include a microphone, a camera, a thermometer, sphygmomanometer, a Holter monitor, and/or an electroencephalography (EEG) device. In select implementations, the biometric sensors are included in a user device, such as a smartwatch or smartphone. The biometric sensors can measure an inflection of the voice of the call taker, a tone of the voice of the call taker, a pace of speech of the call taker, a breathing rate of the call taker, a volume of the voice of the call taker, a blood pressure of the call taker, a heart rate of the call taker, and/or a temperature of the call taker.

The electronic air freshener 130 can be any electronically-controlled device that can alter an attribute of the air. For example, in some implementations, the electronic air freshener 130 can be or include a device that adds a scent to the air, such as an electric incense burner, a battery-operated candle, or a scent diffuser. In select implementations, the electronic air freshener 130 can be or include a humidifier, a dehumidifier, an air cleaner, and/or an air purifier.

The display 140 can be or include a screen. The display 140 can be or include a liquid-crystal display, a plasma display, a cathode ray tube, a video projector, a rear-projector, a digital light projector, a liquid crystal on silicon display, an organic LED (light-emitting diode), a microLED, a field-emission display, and/or a surface-conduction electron-emitter display. The display 100 can perform one or more operations in the call center 100. For example, the display 140 can display a current day, date, time, and/or temperature/weather. The display 140 can display a list of call takers currently in the call canter. The display 140 can display a number of calls in a call queue. The display 140 can display a map of a jurisdiction served by the call center 100. In particular implementations of the present disclosure, the display 140 can display positive images, such as cute photographs or motivational messages to reduce call taker stress.

The fan 150 can be a ceiling fan and is merely an illustration representing one of a greater class of heating, ventilation, and air conditioning (HVAC) devices. For example, the fan 150 can be, include, or be substituted with a heater, a chiller, air handler, a blower, or air distributor.

The temperature control 160 can include a thermometer. The temperature control 160 can operate as a front-end to the electronic air freshener 130 and/or the fan 150. Thus, in various implementations, the temperature control 160 can be operated by a human to modify the air in the call center 100.

The cot 170 illustratively represents a break area in the call center 100. Thus, the cot 170 can be, include, or be substituted with a chair (including a recliner and/or a massage recliner), a break room (particularly including a place to eat and/or drink), and/or an exercise room (including free weights, workout machines, or yoga pads).

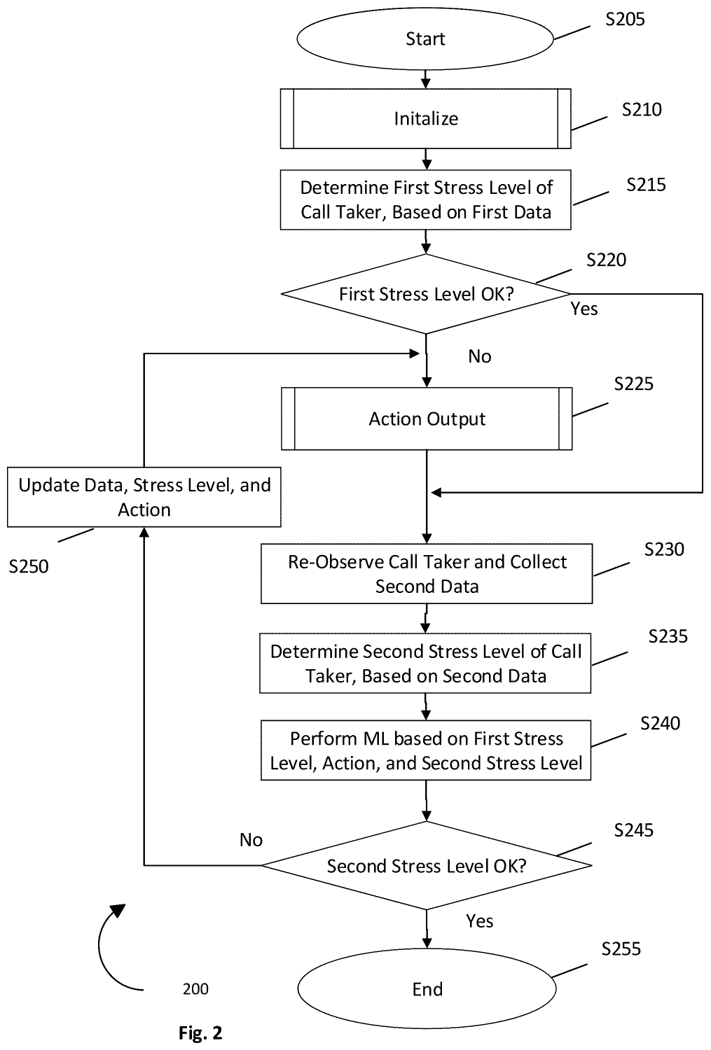

FIG. 2 illustrates an exemplary stress recognition algorithm 200, according to an implementation of the present disclosure. The algorithm 200 begins at S205 and advances to S210.

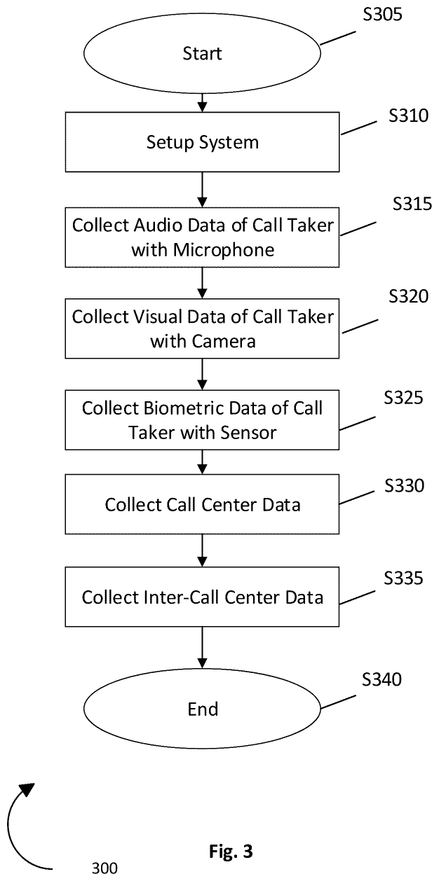

In S210, the algorithm 200 performs an initialization operation to obtain first data. The initialization operation is discussed in more detail in FIG. 3. The algorithm 200 then advances to S215.

In S215, the algorithm 200 determines a first stress level of a call taker, based on the first data.

For example, the algorithm 200 determines the first stress level based on the audio data, the visual data, the biometric data, the call center data and/or the inter-call center data. That is, the first stress level can be determined based on the call taker's voice inflection, as just one example. A person having ordinary skill in the art would understand how to determine a stress level revealed in a call taker's voice inflection or pulse rate, for example. The algorithm can produce the first stress level in the form of a scalar. In some implementations, the scalar is in a relative range, such as 1-100. In various implementations, the scalar is an absolute number, such as beginning at 1 with no particular upper bound. In select implementations, the first stress level is produced in a more qualitative manner, such as by assigning a label (e.g., “low,” “medium,” or “high”) by a machine-learning model.

Further, the algorithm 200 can retrieve an incident type of a call handled by the call taker. Thus, given a recentness (e.g., based on a current date/time, as well as a date/time of the call), frequency, and/or distribution of incident types of calls taken by the call taker, the system can direct the call taker to rotate out of the phone queue.

The limit can be, for example, after the third homicide. The period can be a shift or twenty-four hours.

The algorithm 200 then advances to S220.

In S220, the algorithm 200 determines whether the first stress level of the call taker is OK or within an acceptable normal range.

In many implementations, this determination is based on absolute stress levels (e.g., without considering how this particular call taker handles stress). For example, in an implementation in which the first stress level is a scalar, the algorithm 200 can determine whether the scalar exceeds a predetermined value (e.g., 80). In an implementation in which the first stress level is a label, the algorithm 200 can determine whether the label indicates more than a predetermined stress level (e.g., “medium-high”). If the algorithm 200 determines that the first stress level of the call taker is OK, then the algorithm 200 advances to S230. If the algorithm 200 determines that the first stress level of the call taker is not OK, then the algorithm 200 advances to S225.

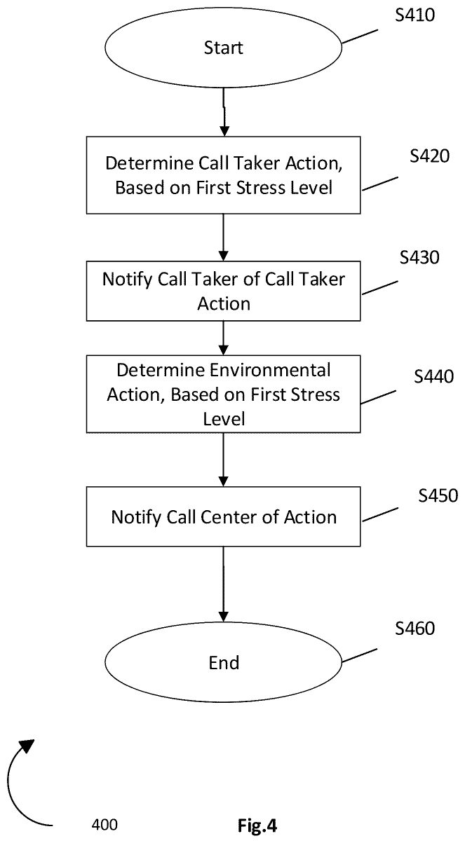

In S225, the algorithm 200 produces an action output. The action outputs are discussed in more detail with regard to FIG. 4. The algorithm 200 then advances to S230.

In S230, the algorithm 200 re-observes the call taker and collects second data. In many implementations, the second data are of the same type as the first data described in FIG. 3 (e.g., can be collected via the sensors described with reference to S315-S335) below. Thus, in various implementations, the second data can reflect how attributes of the call taker vary across time. For example, the second data can reflect if and how the facial features of the call taker have changed from the action output in S225.

Further, in some implementations, the second data can be obtained via a slightly different collection than in S315-S335. For example, a particular implementation might not obtain facial features in S210 for the first data but does obtain them for the second data in S230.

The algorithm 200 then advances to S235.

In S235, the algorithm 200 determines a second stress level of the call taker, at least in part based on the second data. For example, the algorithm 200 can determine the second stress level based on the audio data, the visual data, the biometric data, the call center data and/or the inter-call center data collected in S230. That is, the second stress level can be determined based on the call taker's voice inflection, as just one example. The algorithm can produce the second stress level in the form of a scalar. In some implementations, the scalar is in a relative range, such as 1-100. In various implementations, the scalar is an absolute number, such as beginning at 1 with no particular upper bound. In select implementations, the second stress level is produced in a more qualitative manner, such as by assigning a label (e.g., “low,” “medium,” or “high”) by a machine-learning model.

The algorithm 200 then advances to S240.

In S240, the algorithm 200 performs machine learning, at least in part based on the first stress level, the output action, and the second stress level. The machine learning can be performed via a neural network, for example. Alternatively or additionally, the machine learning can be performed via random forests, support vector machines, a k-nearest neighbors algorithm, and symbolic regression.

The machine learning can be trained on a big data set including human responses to call taker actions and environmental actions. The call taker actions and environmental actions can relate to the call taker actions and environmental actions discussed in the context of FIG. 4 below. Especially in this context, the phrases “call taker actions” and “environmental actions” are not limited to actions performed by a call taker nor in a call center environment. For example, the action of going for a walk can be performed by someone other than a call taker. The environmental actions can be, for example, displaying more than a de minimis amount of a blue color. Thus, the big data set can be based on a broader category, such as “human,” rather than a narrow category, such as “call taker.”

The sensors used in S230 can be the same as in S315, S320, and S325, and the stress level determination in S235 can be the same as in S215. However, the sensors and stress level determination can differ. For example, by assessing a call taker's stress level with different sensors, the system can reflect relative processing times. For example, the determination in S215 can be based on blood pressure, which typically involves less processing time. In contrast, the determination in S235 can be based on video data, which typically uses more processing time than a blood pressure determination. Thus, the system can effectively front-load the stress determination to begin reducing stress earlier, with more diverse (and potentially more accurate) processing waiting until later.

The algorithm 200 then advances to S245.

In S245, the algorithm 200 determines whether the second stress level of the call taker is OK. In a few implementations, this determination is based on absolute stress levels (e.g., without considering how this particular call taker handles stress). For example, in an implementation in which the second stress level is a scalar, the algorithm 200 can determine whether the scalar is below a predetermined value (e.g., 65). In an implementation in which the second stress level is a label, the algorithm 200 can determine whether the label indicates less than a predetermined stress level (e.g., “medium-low”).

In many implementations, this determination is based on relative stress levels. For example, the determination can be based on previous stress levels and/or sensor readings for the specific call taker. In particular, this determination can be based on a comparison with the first stress level determined in S215 and, especially, a difference between the first stress level determined in S215 and the second stress level in S235.

In some implementations in which the determination considers the difference between the first stress level and the second stress level, the algorithm 200 can pause in S245 to see if the difference continues to increase in magnitude. This pause can avoid situations in which the stress level is decreasing at a rate slower than reflected by the system. Thus, exposing the call taker to additional changes might increase or, at least, render less effective some of the existing changes to reduce stress.

If the algorithm 200 determines that the second stress level of the call taker is not OK, then the algorithm 200 advances to S250.

In S250, the algorithm 200 updates the data and the stress level of the call taker. In select implementations, this update replaces the first data with the second data and replaces the first stress level with the second stress level. In many implementations, a history of data and stress levels is maintained, such that, for example, pointers to the first data and the first stress level are updated to point to the second data and the second stress level.

In addition, the algorithm 200 can update the action output (e.g., in S225), based on the machine learning. For example, if a previous action output did not effectively lower the stress level of the call taker, then the algorithm 200 is less likely to recommend the same action output for the same call taker (or another call taker). On the other hand, if a previous action output did effectively lower the stress level of the call taker, the algorithm 200 is more likely to recommend the same action output or more of the same action output for the call taker (or another call taker).

In some implementations, this action output can vary in intensity. For example, a three-minute walk did not lower the stress level of the call taker. Thus, the algorithm might reduce the likelihood of recommending a three-minute walk to this call taker but might increase the likelihood of recommending a five-minute walk to the call taker (especially if walks seem effective among other call takers in the same call center). On the other hand, if recommending a three-minute walk was effective for this call taker, the system can increase the likelihood of recommending walks to this call taker, especially those of longer duration, if the call taker's stress becomes elevated again. The complexity of these relationships (e.g., whether walk itself is ineffective or merely the duration of the walk) is particularly suited for machine learning and big data.

The algorithm 200 then returns to S225, where an output action can be performed, based on what was previously the second stress level.

Returning to S245, if the algorithm 200 determines that the second stress level of the call taker is OK, then the algorithm 200 advances to S255, in which the algorithm 200 concludes.

FIG. 3 illustrates suboperations of an initialization operation 300, according to an implementation of the present disclosure. The algorithm for the initialization operation 300 begins at S305 and advances to S310.

In S310, the system is set up for a call taker. This set up can include receiving attributes of a particular call taker, such as gender or sex, weight, and/or height. In some implementations, the set up can include a nominal heart rate, such as before a work shift of the call taker. In many implementations, this information is retrieved from a memory, based on a name and/or an employee number of the call taker, for example. The algorithm for the initialization operation 300 then advances to S315.

In S315, the algorithm collects audio data of the call taker using a microphone. The audio data can include, for example, the call taker's voice inflection, their tone of voice, their rate of speech, the volume of their voice, and their breathing rate. In many implementations, the microphone is the same microphone used by the call taker to participate in emergency calls (e.g., a microphone in a headset worn by the call taker). In some implementations, the system includes an additional microphone to record the audio data.

The algorithm for the initialization operation 300 then advances to S320.

In S320, the algorithm collects visual data (e.g., photographic or video data) of the call taker using a camera. The visual data can include, for example, the facial features of the call taker, including their facial expression, eye activity, mouth activity, and head movements. The facial expression can include, for example, the presence of forehead wrinkles or a downturned mouth. The algorithm for the initialization operation 300 then advances to S325.

In S325, the algorithm collects biometric data of the call taker using a sensor. The biometric data can be or include the blood pressure of the call taker, the heart rate of the call taker, an electrocardiogram of the heart of the call taker, a temperature of the call taker, and/or an electrogram of the brain of the call taker. In many implementations, this data is collected via dedicated sensors, such as a sphygmomanometer to measure blood pressure, a Holter monitor for an electrocardiogram, a thermometer for the temperature, or an electroencephalography (EEG) device for the electrogram of the brain. In some implementations, the data can be collected via a more general sensor: for example, some cameras can approximate the call taker's temperature. In addition, the data can be collected by a user device, such as a smartwatch or smartphone.

In various implementations, equipment present in the call center can be enhanced to provide appropriate functionality. For example, a headset of the call taker can be enhanced to include an electrode for producing an electrogram.

In addition, in select implementations, the audio or visual data can provide biometric data. For example, the call taker's breathing rate can be considered biometric data.

The algorithm for the initialization operation 300 then advances to S330.

In S330, the algorithm collects call center data. The call center data can include a call volume (e.g., a number of calls) taken by the call taker and/or a severity of the calls taken by the call taker, for example. In select implementations, the call center data can include a number of hours worked by the call taker during a predefined period (e.g., a day or a week) and/or the number of calls per period. Further, the call center data can include a period since the call taker's last break, as well as a ratio of break time to shift duration.

In addition, the algorithm 300 can collect an incident type for each call taken by the call taker. For example, at the conclusion of the call taker's handling of each call or of the incident underlying the call, the algorithm can code the call with an incident type. In some implementations, the incident type can be assigned by the Emergency Call Tracking System (ECaTS) produced by Intrado.

For example, the incident type can be one or more of e.g., homicide, children involved, domestic abuse, car accident, fatality, birth, theft, extreme weather, property damage, or fire. Thus, some implementations recognize that some types of calls (e.g., homicide) can be more stressful than others (e.g., property damage). Other incident types are possible, as well.

When the call taker handles a call, the algorithm 300 can store in a memory an indication of the call taker (e.g., name and/or employee number) in association with the type of the call and the time/date of the call. Thus, when initializing the system for the call taker (e.g., in S310), the system can determine the incident types of calls handled by the particular call taker, as well as the times/dates of the calls.

The algorithm for the initialization operation 300 then advances to S335.

In S335, the algorithm 300 collects inter-call center data. Various implementations of the algorithm 300 are performed at a per-call-taker basis. That is, because an individual's features might vary from an average or otherwise idealized call taker, a particular implementation might be tailored to the rate of speech, facial features, or biometric data of the individual. In so doing, the granularity of the algorithm 300 can address practices of particular individuals.

For example, because the demand for qualified call takers is so high, individuals have been known to work at multiple call centers. Although the call center data collected in S330 might reflect the number of calls taken by an individual within a particular call center during a particular week, that data might not reflect the number of calls taken by the individual in another call center during the same week. Therefore, the algorithm 300 advantageously can manage information from other call centers to track data by an individual in another call center. Alternatively or in addition, a request can be sent to another call center (or, more accurately, a computer managing its data) for data about an individual. For example, the request can include an identifier (e.g., a name) of the individual and a duration, and a reply can be received that indicates the number of hours worked by the individual at the other call center during the duration.

The algorithm for the initialization operation 300 then advances to S340, in which the algorithm concludes.

FIG. 4 illustrates suboperations 400 of an action output operation, according to an implementation of the present disclosure. The suboperations 400 begin at S410 and advance to S420.

In S420, the suboperations 400 optionally determine a call taker action, at least in part based on the first stress level of the call taker. For example, the call taker action can be or include taking a break, eating a healthy snack, drinking a non-alcoholic beverage (such as water or a sports drink), taking a walk, listening to music, taking a nap, doing exercise (especially, at the call taker's desk), taking a dance break, and/or playing a game.

In select implementations, the severity of the first stress level forms a basis for the determination of the call taker action. In various implementations, the severity can be based on how much the first stress level exceeds a predetermined value (e.g., 85) or predetermined stress level (e.g., “medium low”).

In some instances, a call taker action that relieves more stress is potentially more disruptive to the function of the call center than a call taker action that relieves less stress. For example, although a nap might relieve more stress than a water break, a nap might remove the call taker from the call queue for 15 minutes or more, while a water break might remove the call taker from the call queue for 3 minutes or less. Thus, many implementations manage call taker stress levels, rather than minimize call taker stress levels.

Accordingly, the system can reserve actions that are expected to relieve a greater amount of stress (e.g., taking a walk) for when the severity of the first stress level is high. Similarly, the system can recommend actions that are expected to relieve a lesser amount of stress (e.g., eating a snack or switching to administrative duties), if the severity of the first stress level is low. In some implementations, the system sets a limit for particular call types, after which, e.g., the call taker is removed from the phone queue.

In select implementations, the call taker action additionally can be based on the biometric data of the call taker. For example, if the call taker's hydration level is low or if their blood sugar is low, the call taker action is more likely to be a water break or a snack, respectively. On the other hand, if the call taker's hydration level or blood sugar is adequate, then the call taker action is less likely to be a water break or a snack, respectively. In many implementations, the system can record the call taker action.

The suboperations 400 then advance to S430.

In S430, the suboperations 400 notify the call taker of the call taker action. For example, the notification can be performed visually, such as via a message displayed on a monitor of the call taker. In some implementations, the notification can be performed audibly, such as via a message sent to the call taker's headset. In select implementations, the notification can be performed in other ways, such as via a text message sent to the call taker's mobile phone.

The suboperations 400 then advance to S440.

In S440, the suboperations 400 optionally determine an environmental action, at least in part based on the first stress level. The environmental actions can affect more than one call taker. For example, the environmental action can be or include displaying and/or updating a leaderboard, changing a color of light (e.g., overhead lights or desktop lights to blue) and/or on a display, changing a refresh rate for fresh air, introducing a pleasant scent, changing an air temperature, and/or displaying a positive (e.g., cute and/or inspirational) image on a display.

The refresh rate can, in part, be based on an air quality sensor. For example, if the air quality is low, then the system is more likely to recommend increasing the air refresh rate. On the other hand, if the air quality is high, then the system is less likely to recommend increasing the air refresh rate.

The pleasant scent can be introduced by activating an electronic air freshener (e.g., Glade PlugIn or Air Wick warmer).

The suboperations 400 then advance to S450.

In S450, the system notifies the call center of the call taker action determined in S420 and/or the environmental action determined in S440.

In some implementations, the notification can be to a computer of the call center. For example, the computer can manage the queue based on the call taker action by removing the call taker from the queue to permit the call taker to take a nap. Alternatively or in addition, the computer can manage the environment of the call center, such as by modifying the lighting or the heating, ventilation, and air conditioning (HVAC).

The computer can also display on a monitor a leader board for lower stress levels, indicators thereof (e.g., heart rate), or stress levels to encourage call takers to participate.

In various implementations, the notification can be intended for a human (e.g., a call center manager or supervisor) in the call center, notwithstanding the involvement of a computer. For example, the notification can be performed visually, such as via a message displayed on a monitor of the manager or supervisor. In some implementations, the notification can be performed audibly, such as via a message sent to a headset of the manager or supervisor. In select implementations, the notification can be performed in other ways, such as via a text message sent to a mobile phone of the manager or supervisor. Thus, the call center manager or supervisor can understand why a particular call taker might not be at their desk, on administrative duties, or on break. Thus, in case a surge occurs, the call center manager or supervisor can make a decision about the tasks performed by a particular call taker.

The notification also can indicate a prize should be awarded to a particular call taker for effective stress management or reduction.

The suboperations 400 then advance to S460, in which the suboperations 400 conclude.

It is also possible to measure cortisol levels in S210 and S230 using, for example, saliva of a call taker. Because this measure is more intrusive, it might be less frequently and can be used to supplement existing data.

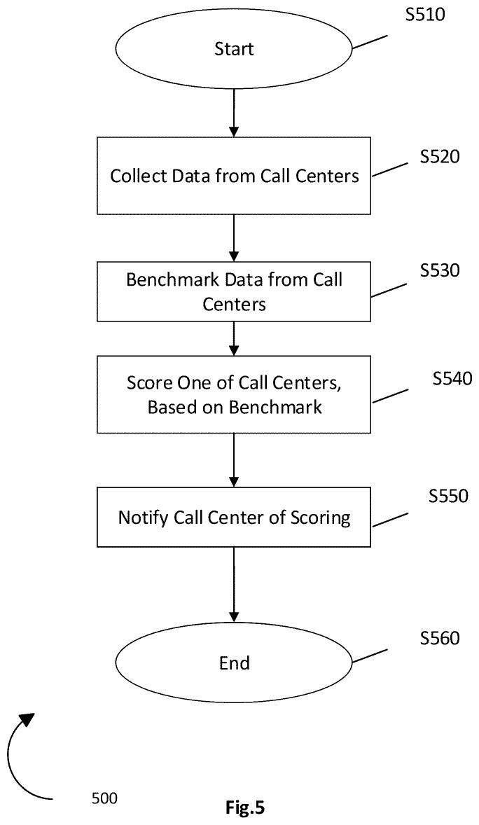

FIG. 5 illustrates an algorithm 500 for call taker behavior modification, based on big data collection, according to an implementation of the present disclosure.

Above, data (e.g., the first data collected in S215 and the second data collected in S235) is collected, and based thereon, an individual or environment is directed for modification of individual behaviors on a per-call-taker basis.

The algorithm 500 begins at S510 and advances to S520. In S520, the algorithm 500 collects the first data and the second data from a plurality of call centers. These call centers can be set across one or more geographical divisions, such as a governmental jurisdiction (e.g., nation, state, county, city), a population center (e.g., an urban area), or cultural area (e.g., Appalachia). The algorithm 500 then advances to S530.

In S530, the algorithm 500 performs benchmarking for a particular geographical division, based on the data received in S520. The benchmarking can consider any biometric data, such as blood pressure, heart rate, or breathing rate. The benchmarking can include any statistical analysis, such as averaging, determining a median or mode, determining a standard deviation, and so on.

In many implementations, the benchmarking is performed at various granularities of the one or more geographical divisions. For example, benchmarking can be performed at a national granularity level, based on call centers within a nation; at a state granularity level, based on call centers within a state of the nation; and at a county granularity level, based on call centers within the county of the state. Thus, the algorithm 500 can use the data of each of the plurality of call centers at a plurality of granularities, based on the geographical divisions (e.g., a county, a state, and a nation) to which the individual call center belongs.

The algorithm 500 then advances to S540.

In S540, the algorithm 500 produces a respective score of each of the plurality of call centers against the benchmarks. The scoring can be on a scale of 1-100, where 50 is average, for example. The scoring can also be on a non-numeric scale, such as an alphabetic grade rating (e.g., A-F, where C is average) or a color system (e.g., a stoplight color system, where yellow is average). The scoring can be based, in some implementations, on the call type. The algorithm 500 then advances to S550.

In S550, the algorithm 500 notifies each of the plurality of call centers of the scoring produced in S540. The algorithm 500 then advances to S560 and concludes.

Thus, with the algorithm 500, it becomes possible to determine whether call takers at call centers in cities are more or less stressed than in a rural setting. In addition, the algorithm 500 can determine the acceptable level of stress throughout a large jurisdiction (e.g., the country), as well as how long can a call taker maintain a peak stress level until recommending a stress-reducing action.

Thus, an implementation can provide a big data system for this data and a set of more vetted remediations based on having enhanced visibility.

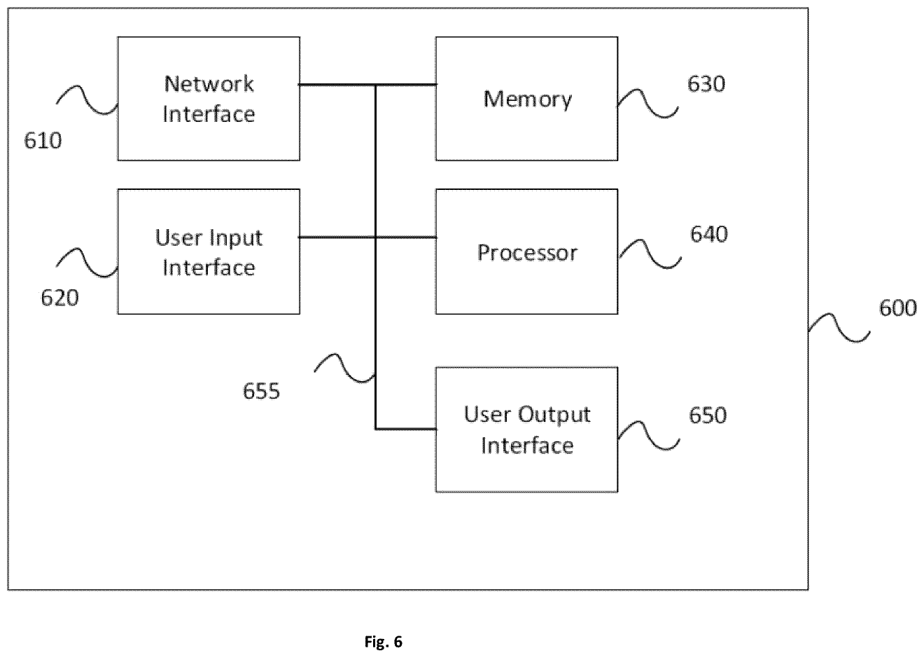

FIG. 6 illustrates a computing device 600, according to an implementation of the present disclosure. The computing device 600 can include a network interface 610, a user input interface 620, a memory 630, a processor 640, a user output interface 650, and a bus 655.

Although illustrated within a single housing, the computing device 600 can be distributed across plural housings or sub-systems that cooperate in executing program instructions. In some implementations, the computing device 600 can include one or more blade server devices, standalone server devices, personal computers (including laptop computers and tablet computers), routers, hubs, switches, bridges, firewall devices, intrusion detection devices, mainframe computers, network-attached storage devices, smartphones and other mobile telephones, and other computing devices. Although the system executes the Windows OS and/or macOS in many implementations, the system hardware can be configured according to a Symmetric Multi-Processing (SMP) architecture or a Non-Uniform Memory Access (NUMA) architecture.

The network interface 610 that provides one or more communication connections and/or one or more devices that allow for communication between the computing device 600 and other computing systems (not shown) over a communication network, collection of networks (not shown), or the air. The network interface can communicate using near-field communications (NFC), Wi-Fi™, Bluetooth, Ethernet, cellular (e.g., 5G), facsimile, or any other wired or wireless interface.

The user input interface 620 that can receive one or more inputs from a human. The user input interface can be or include a mouse, a touchpad, a keyboard, a touchscreen, a trackball, a camera, a microphone, a joystick, a game controller, a scanner, or any other input device.

The memory 630, also termed a “storage,” can include or be one or more computer-readable storage media readable by a processor 640 and that store software. The memory 630 can be implemented as one storage device and can also be implemented across multiple co-located or distributed storage devices or sub-systems. The memory 630 can include additional elements, such as a controller, that communicate with the processor 640. The memory 630 can also include storage devices and/or sub-systems on which data and/or instructions are stored. The computing device 600 can access one or more storage resources to access information to carry out any of the processes indicated in this disclosure and, in particular, FIGS. 2-5. In various implementations, the memory 630 stores software to execute at least a portion of the algorithms illustrated in FIGS. 2-5. Further, the software, when executed by the computing device 600 generally and/or the processor 640 specifically, can direct, among other functions, performance of the operations as described herein.

The memory 630 can be or include a read-only memory (ROM), a programmable ROM (PROM), an erasable PROM (EPROM), an electrically erasable PROM (EEPROM), a random-access memory (RAM), a dynamic RAM (DRAM), a static RAM (SRAM), a hard drive, a cache memory, a flash memory, a removable disk, or a tape reel. The memory 630 can be or include resistive RAM (RRAM) or a magneto-resistive RAM (MRAM).

The processor 640 (e.g., a processing unit) can be or include one or more hardware processors and/or other circuitry that retrieve and execute software from the memory 630. The processor 640 can be implemented within one processing device, chip, or package and can also be distributed across multiple processing devices, chips, packages, or sub-systems that cooperate in executing program instructions. In some implementations, the processor 640 is or includes a Graphics Processing Unit (GPU).

The processor 640 can have any register size, such as a 32-bit register or a 64-bit register, among others. The processor 640 can include multiple cores. Implementations of the processor 640 are not limited to any particular number of threads. The processor 640 can be fabricated by any process technology, such as 14 nm process technology.

The computing device 600 can also include a user output interface 650 that outputs information to a human user. The user output interface 650 can be or include a display (e.g., a screen), a touchscreen, speakers, a printer, or a haptic feedback unit. In many implementations, the user output interface 650 can be combined with the user input interface 620. For example, some implementations include a touchscreen or headset including headphones and a microphone.

In implementations including multiple computing devices, a server of the system or, in a serverless implementation, a peer can use one or more communications networks that facilitate communication among the computing devices. For example, the one or more communications networks can include or be a local area network (LAN) or wide area network (WAN) that facilitate communication among the computing devices. One or more direct communication links can be included between the computing devices. In addition, in some cases, the computing devices can be installed at geographically distributed locations. In other cases, the multiple computing devices can be installed at one geographic location, such as a server farm or an office.

As used herein, the terms “storage media” or “computer-readable storage media” can refer to non-transitory storage media, such as non-limiting examples of a hard drive, a memory chip, and cache memory, and to transitory storage media, such as carrier waves or propagating signals.

Aspects of the system can be implemented in various manners (e.g., as a method, a system, a computer program product, or one or more computer-readable storage media). Accordingly, aspects of the present disclosure can take the form of a hardware implementation, a software implementation (including firmware, resident software, or micro-code) or an implementation combining software and hardware aspects that can generally be referred to herein as a “circuit,” “module” or “system.” Functions described in this disclosure can be implemented as an algorithm executed by one or more hardware processing units, e.g., one or more microprocessors of one or more computers. In various embodiments, different operations and portions of the operations of the algorithms described can be performed by different processing units. Furthermore, aspects of the present disclosure can take the form of a computer program product embodied in one or more computer-readable media having computer-readable program code embodied, e.g., encoded or stored, thereon. In various implementations, such a computer program can, for example, be downloaded (or updated) to existing devices and systems or be stored upon manufacture of these devices and systems.

The innovations in this detailed description can be implemented in a multitude of different ways, for example, as defined and covered by the claims and/or select examples. In the description, reference is made to the drawings where like reference numerals can indicate identical or functionally similar elements. Elements illustrated in the drawings are not necessarily drawn to scale. Additionally, certain implementations can include more elements than illustrated in a drawing and/or a subset of the elements illustrated in a drawing. Further, some implementations can incorporate a suitable combination of features from two or more drawings.

The disclosure describes various illustrative implementations and examples for implementing the features and functionality of the present disclosure. The components, arrangements, and/or features are described in connection with various implementations and are merely examples to simplify the present disclosure and are not intended to be limiting. In the development of actual implementations, implementation-specific decisions can be made to achieve specific goals, including compliance with system, business, and/or legal constraints, which can vary from one implementation to another. Additionally, while such a development effort might be complex and time-consuming, it would be a routine undertaking for those of ordinary skill in the art having the benefit of this disclosure.

The systems, methods and devices of this disclosure have several innovative aspects, no one of which is solely responsible for the attributes disclosed herein. Some objects or advantages might not be achieved by implementations described herein. Thus, for example, certain implementations can operate in a manner that achieves or optimizes one advantage or group of advantages as taught herein and not other objects or advantages as taught or suggested herein.

In one example implementation, electrical circuits of the drawings can be implemented on a board of an associated electronic device. The board can be a general circuit board that can hold various components of the internal electronic system of the electronic device and, further, provide connectors for other peripherals. More specifically, the board can provide the electrical connections by which other components of the system can communicate electrically. Any processors (inclusive of digital signal processors, microprocessors, supporting chipsets, etc.) and computer-readable non-transitory memory elements can be coupled to the board based on configurations, processing demands, and computer designs. Other components such as external storage, additional sensors, controllers for audio/video display, and peripheral devices can be attached to the board as plug-in cards, via cables, or integrated into the board itself. In various implementations, the functionalities described herein can be implemented in emulation form as software or firmware running within one or more configurable (e.g., programmable) elements arranged in a structure that supports these functions. A non-transitory, computer-readable storage medium can include instructions to allow one or more processors to carry out the emulation.

In another example implementation, the electrical circuits of the drawings can be implemented as stand-alone modules (e.g., a device with associated components and circuitry configured to perform a specific application or function) or implemented as plug-in modules into application specific hardware of electronic devices. Implementations of the present disclosure can be readily included in a system-on-chip (SOC) package. An SOC represents an integrated circuit (IC) that integrates components of a computer or other electronic system into one chip. The SOC can contain digital, analog, mixed-signal, and often radio frequency functions on one chip substrate. Other implementations can include a multi-chip-module (MCM), with a plurality of separate ICs located within one electronic package and that interact through the electronic package. In various other implementations, the processors can be implemented in one or more silicon cores in Application Specific Integrated Circuits (ASICs), Field Programmable Gate Arrays (FPGAs), programmable array logic (PAL), generic array logic (GAL), and other semiconductor chips.

The specifications, dimensions, and relationships outlined herein (e.g., the number of processors and logic operations) have been offered for non-limiting purposes of example and teaching. Such information can be varied considerably. For example, various modifications and changes can be made to arrangements of components. The description and drawings are, accordingly, to be regarded in an illustrative sense, not in a restrictive sense.

The numerous examples provided herein described interaction in terms of two, three, or more electrical components for purposes of clarity and example. The system can be consolidated in any manner. Along similar design alternatives, the illustrated components, modules, and elements of the drawings can be combined in various possible configurations within the scope of this disclosure. In certain cases, one or more of the functionalities of a given set of flows might be more clearly described by referencing a limited number of electrical elements. The electrical circuits of the drawings and their teachings are readily scalable and can accommodate many components, as well as more complicated/sophisticated arrangements and configurations. Accordingly, the provided examples do not limit the scope or inhibit the teachings of the electrical circuits as potentially applied to a myriad of other architectures.

In this disclosure, references to various features (e.g., elements, structures, modules, components, steps, operations, characteristics, etc.) included in “one implementation,” “example implementation,” “an implementation,” “another implementation,” “some implementations,” “various implementations,” “other implementations,” “alternative implementation,” and the like are intended to mean that any such features can be included in one or more implementations of the present disclosure and might or might not necessarily be combined in the same implementations. Some operations can be deleted or omitted where appropriate, or these operations can be modified or changed considerably. In addition, the timing of these operations can be altered considerably. The preceding operational flows have been offered for purposes of example and discussion. Implementations described herein provide flexibility in that any suitable arrangements, chronologies, configurations, and timing mechanisms can be provided.

### EXAMPLES

In Example M1, a method includes observing a call taker to produce first data; determining a first stress level of the call taker, at least in part based on the first data; determining if the first stress level exceeds a first predetermined threshold; determining an output action, if the first stress level exceeds the first predetermined threshold; and notifying of the action.

Example M2 is the method of Example M1, further including: observing the call taker to produce second data; determining a second stress level of the call taker, at least in part based on the second data; determining if the second stress level is less than a second predetermined threshold; and updating the first data and the first stress level, at least in part based on the second data and the second stress level, if the second stress level is not less than the second predetermined threshold.

Example M3 is the method of Example M2, further including: performing machine learning, at least in part based on the first stress level, the action, and the second stress level; and outputting an action, at least in part based on the machine learning.

Example M4 is the method of any of Examples M1-M3, further including: setting up the system with a heart rate of the call taker.

Example M5 is the method of any of Examples M1-M4, wherein the output action is environmental.

Example M6 is the method of any of Examples M1-M5, wherein the output action is for the call taker.

Example M7 is the method of any of Examples M1-M6, wherein the first data and the second data include audio data, visual data, or biometric data.

In Example A1, an apparatus includes a processor configured to produce first data from observations of a call taker, to determine a first stress level of the call taker, at least in part based on the first data, to determine if the first stress level exceeds a first predetermined threshold, to determine an output action, if the first stress level exceeds the first predetermined threshold, and to notify of the action.

Example A2 is the apparatus of Example A1, wherein the processor further is configured to produce second data from observations of the call taker to produce second data, to determine a second stress level of the call taker, at least in part based on the second data, to determine if the second stress level is less than a second predetermined threshold, and to update the first data and the first stress level, at least in part based on the second data and the second stress level, if the second stress level is not less than the second predetermined threshold.

Example A3 is the apparatus of Example A2, wherein the processor further is configured to perform machine learning, at least in part based on the first stress level, the action, and the second stress level, and to output an action, at least in part based on the machine learning.

Example A4 is the apparatus of any of Examples A1-A3, wherein the first data includes a blood pressure or heart rate of the call taker.

Example A5 is the apparatus of any of Examples A1-A4, wherein the output action is environmental.

Example A6 is the apparatus of any of Examples A1-A5, wherein the output action is for the call taker.

Example A7 is the apparatus of any of Examples A1-A6, wherein the first data and the second data include audio data or visual data of the call taker.

In Example C1, a non-transitory, computer-readable medium including instructions that, when executed by a processing unit, perform operations including producing first data from observations of a call taker; determining a first stress level of the call taker, at least in part based on the first data; determining if the first stress level exceeds a first predetermined threshold; determining an output action, if the first stress level exceeds the first predetermined threshold; and notifying of the action.

Example C2 is the medium of Example C1, the operations further comprising: producing second data from observations of the call taker to produce second data; determining a second stress level of the call taker, at least in part based on the second data; determining if the second stress level is less than a second predetermined threshold; and updating the first data and the first stress level, at least in part based on the second data and the second stress level, if the second stress level is not less than the second predetermined threshold.

Example C3 is the medium of Example C2, the operations further comprising: performing machine learning, at least in part based on the first stress level, the action, and the second stress level; and outputting an action, at least in part based on the machine learning.

Example C4 is the medium of any of Examples C1-C3, wherein the first data includes a blood pressure of the call taker.

Example C5 is the medium of any of Examples C1-C4, wherein the output action is environmental.

Example C6 is the medium of any of Examples C1-C5, wherein the output action is for the call taker.

Example C7 is the medium of any of Examples C1-C6, wherein the first data and the second data include audio data or visual data of the call taker.

In Example F1, an apparatus includes processing means for producing first data from observations of a call taker, for determining a first stress level of the call taker, at least in part based on the first data, for determining if the first stress level exceeds a first predetermined threshold, for determining an output action, if the first stress level exceeds the first predetermined threshold, and for notifying of the action.

Example F2 is the apparatus of Example F1, wherein the processing means further is for producing second data from observations of the call taker to produce second data, for determining a second stress level of the call taker, at least in part based on the second data, for determining if the second stress level is less than a second predetermined threshold, and for updating the first data and the first stress level, at least in part based on the second data and the second stress level, if the second stress level is not less than the second predetermined threshold.

Example F3 is the apparatus of Example F2, wherein the processing means further is for performing machine learning, at least in part based on the first stress level, the action, and the second stress level, and for outputting an action, at least in part based on the machine learning.

Example F4 is the apparatus of any of Examples F1-F3, wherein the first data includes a blood pressure or heart rate of the call taker.

Example F5 is the apparatus of any of Examples F1-F4, wherein the output action is environmental.

Example F6 is the apparatus of any of Examples F1-F5, wherein the output action is for the call taker.

Example F7 is the apparatus of any of Examples F1-F6, wherein the first data and the second data include audio data or visual data of the call taker.

In Example AA1, a communication center includes a computer configured to be operated by a call taker; a first sensor configured to observe the call taker to provide first data; and a processor configured to determine a first stress level of the call taker, at least in part based on the first data, to determine if the first stress level exceeds a first predetermined threshold, to determine an output action, if the first stress level exceeds the first predetermined threshold, and to perform a notification of the action.

Example AA2 is the communication center of Example AA1, wherein the notification notifies the computer of the action.

Example AA3 is the communication center of any of Examples AA1-AA2, wherein the first sensor further is configured to measure at least one attribute selected from the group consisting of an inflection of the voice of the call taker, a tone of the voice of the call taker, a pace of speech of the call taker, a breathing rate of the call taker, a volume of the voice of the call taker, a blood pressure of the call taker, a heart rate of the call taker, and a temperature of the call taker.

Example AA4 is the communication center of any of Examples AA1-AA3, wherein the first sensor includes a camera configured to record a facial feature of the call taker.

Example AA5 is the communication center of any of Examples AA1-AA4, further comprising: an actuator configured to change at least one attribute selected from the group consisting of a color of a portion of the communication center, a refresh rate of the air of a portion in the communication center, a scent of a portion of the communication center, and a display of the communication center, wherein the display is configured to display at least one of a positive image and a leaderboard.

Example AA6 is the communication center of any of Examples AA1-AA5, further comprising: a second sensor configured to measure at least an air quality of a portion of the communication center or a temperature of a portion of the communication center.

Example AA7 is the communication center of any of Examples AA1-AA6, wherein the notification recommends the call taker take a break, eat a snack, or perform an exercise or activity.

In Example MM1, a method to be implemented in a communication center includes: observing a call taker to provide first data; determining a first stress level of the call taker, at least in part based on the first data; determining if the first stress level exceeds a first predetermined threshold; determining an output action, if the first stress level exceeds the first predetermined threshold; and notifying of the action.

Example MM2 is the method of Example MM1, wherein the notifying notifies a computer of the call taker of the action.

Example MM3 is the method of any of Examples MM1-MM2, further comprising: measuring at least one attribute selected from the group consisting of an inflection of the voice of the call taker, a tone of the voice of the call taker, a pace of speech of the call taker, a breathing rate of the call taker, a volume of the voice of the call taker, a blood pressure of the call taker, a heart rate of the call taker, and a temperature of the call taker.

Example MM4 is the method of any of Examples MM1-MM3, wherein the first data includes a facial feature of the call taker.

Example MM5 is the method of any of Examples MM1-MM4, further comprising: changing at least one attribute selected from the group consisting of a color of a portion of the communication center, a refresh rate of the air of a portion in the communication center, a scent of a portion of the communication center, and a display of the communication center, wherein the display is configured to display at least one of a positive image and a leaderboard.

Example MM6 is the method of any of Examples MM1-MM5, further comprising: measuring at least an air quality of a portion of the communication center or a temperature of a portion of the communication center.

Example MM7 is the method of any of Examples MM1-MM6, wherein the notification recommends the call taker take a break, eat a snack, or perform an exercise or activity.

In Example CC1, a non-transitory, computer-readable medium includes instructions that, when executed by a processing unit, perform operations including: determining a first stress level of a call taker, at least in part based on first data observed from the call taker; determining if the first stress level exceeds a first predetermined threshold; determining an output action, if the first stress level exceeds the first predetermined threshold; and performing a notification of the action.

Example CC2 is the medium of Example CC1, wherein the notification notifies a computer of the call taker of the action.

Example CC3 is the medium of any of Examples CC1-CC2, the operations further comprising: measuring at least one attribute selected from the group consisting of an inflection of the voice of the call taker, a tone of the voice of the call taker, a pace of speech of the call taker, a breathing rate of the call taker, a volume of the voice of the call taker, a blood pressure of the call taker, a heart rate of the call taker, and a temperature of the call taker.

Example CC4 is the medium of any of Examples CC1-CC3, wherein the first data includes a facial feature of the call taker.

Example CC5 is the medium of any of Examples CC1-CC4, the operations further comprising: changing at least one attribute selected from the group consisting of a color of a portion of a communication center, a refresh rate of the air of a portion in the communication center, a scent of a portion of the communication center, and a display of the communication center, wherein the display is configured to display at least one of a positive image and a leaderboard.

Example CC6 is the medium of any of Examples CC1-CC5, the operations further comprising: processing at least an air quality of a portion of the communication center or a temperature of a portion of the communication center.

Example CC7 is the medium of any of Examples CC1-CC6, wherein the notification recommends the call taker take a break, eat a snack, or perform an exercise or activity.

In Example FF1, a communication center includes: means for observing a call taker to provide first data; and means for determining a first stress level of the call taker, at least in part based on the first data, for determining if the first stress level exceeds a first predetermined threshold, for determining an output action, if the first stress level exceeds the first predetermined threshold, and for performing a notification of the action.

Example FF2 is the communication center of Example FF1, wherein the notification notifies a computer of the call taker of the action.

Example FF3 is the communication center of any of Examples FF1-FF2, further comprising: means for measuring at least one attribute selected from the group consisting of an inflection of the voice of the call taker, a tone of the voice of the call taker, a pace of speech of the call taker, a breathing rate of the call taker, a volume of the voice of the call taker, a blood pressure of the call taker, a heart rate of the call taker, and a temperature of the call taker.

Example FF4 is the communication center of any of Examples FF1-FF3, wherein the means for observing further is for recording a facial feature of the call taker.

Example FF5 is the communication center of any of Examples FF1-FF4, further comprising: means for changing at least one attribute selected from the group consisting of a color of a portion of the communication center, a refresh rate of the air of a portion in the communication center, a scent of a portion of the communication center, and a display of at least one of a positive image and a leaderboard.

Example FF6 is the communication center of any of Examples FF1-FF5, further comprising: means for measuring at least an air quality of a portion of the communication center or a temperature of a portion of the communication center.

Example FF7 is the communication center of any of Examples FF1-FF6, wherein the notifying recommends the call taker take a break, eat a snack, or perform an exercise or activity.

We claim:

1. A method comprising:
observing a call taker to produce first data;
determining a first stress level of the call taker, at least in part based on the first data;
determining if the first stress level exceeds a first predetermined threshold;
determining an output action, if the first stress level exceeds the first predetermined threshold; and
notifying of the action.

2. The method of claim 1, further comprising:
observing the call taker to produce second data;
determining a second stress level of the call taker, at least in part based on the second data;
determining if the second stress level is less than a second predetermined threshold; and
updating the first data and the first stress level, at least in part based on the second data and the second stress level, if the second stress level is not less than the second predetermined threshold.

3. The method of claim 2, further comprising:
performing machine learning, at least in part based on the first stress level, the action, and the second stress level; and
outputting an action, at least in part based on the machine learning.

4. The method of claim 1, further comprising:
setting up the system with a heart rate of the call taker.

5. The method of claim 1, wherein the output action is environmental.

6. The method of claim 1, wherein the output action is for the call taker.

7. The method of claim 1, wherein the first data and the second data include audio data, visual data, or biometric data.

8. An apparatus, comprising:
a processor configured to produce first data from observations of a call taker, to determine a first stress level of the call taker, at least in part based on the first data, to determine if the first stress level exceeds a first predetermined threshold, to determine an output action, if the first stress level exceeds the first predetermined threshold, and to notify of the action.

9. The apparatus of claim 8, wherein the processor further is configured to produce second data from observations of the call taker to produce second data, to determine a second stress level of the call taker, at least in part based on the second data, to determine if the second stress level is less than a second predetermined threshold, and to update the first data and the first stress level, at least in part based on the second data and the second stress level, if the second stress level is not less than the second predetermined threshold.

10. The apparatus of claim 9, wherein the processor further is configured to perform machine learning, at least in part based on the first stress level, the action, and the second stress level, and to output an action, at least in part based on the machine learning.

11. The apparatus of claim 8, wherein the first data includes a blood pressure of the call taker.

12. The apparatus of claim 8, wherein the output action is environmental.

13. The apparatus of claim 8, wherein the output action is for the call taker.

14. The apparatus of claim 8, wherein the first data and the second data include audio data or visual data of the call taker.

15. A non-transitory, computer-readable medium including instructions that, when executed by a processing unit, perform operations comprising:
producing first data from observations of a call taker;
determining a first stress level of the call taker, at least in part based on the first data;
determining if the first stress level exceeds a first predetermined threshold;
determining an output action, if the first stress level exceeds the first predetermined threshold; and
notifying of the action.

16. The medium of claim 15, the operations further comprising:
producing second data from observations of the call taker to produce second data;
determining a second stress level of the call taker, at least in part based on the second data;
determining if the second stress level is less than a second predetermined threshold; and
updating the first data and the first stress level, at least in part based on the second data and the second stress level, if the second stress level is not less than the second predetermined threshold.

17. The medium of claim 16, the operations further comprising:
performing machine learning, at least in part based on the first stress level, the action, and the second stress level; and
outputting an action, at least in part based on the machine learning.

18. The medium of claim 15, wherein the first data includes a blood pressure of the call taker.

19. The medium of claim 15, wherein the output action is environmental.

20. The medium of claim 15, wherein the first data and the second data include audio data or visual data of the call taker.
